(19) United States
(12) Patent Application Publication
(10) Pub. No.: US 2019/0111174 A1
(43) Pub. Date: Apr. 18, 2019

(54) METHOD FOR STERILIZING OPHTHALMIC LENS

(71) Applicant: HON HAI PRECISION INDUSTRY CO., LTD., New Taipei (TW)

(72) Inventors: HSIU-WEN CHIEN, New Taipei (TW); JIAN-CHIAO WANG, New Taipei (TW)

(21) Appl. No.: 15/802,467

(22) Filed: Nov. 3, 2017

(30) Foreign Application Priority Data
Oct. 17, 2017 (TW) 106135566

(51) Int. Cl. A61L 12/06; B65B 25/00; A45C 11/00

(52) U.S. Cl. CPC A61L 12/063; B29D 11/00067; A45C 11/005; B65B 25/008

(57) ABSTRACT

A method for sterilizing an ophthalmic lens with long-term inhibition against regrowth of micro-organisms includes the step of placing the ophthalmic lens in a sealed container containing a care solution. A photocatalytic agent coated in an inner surface of the container is exposed to ultraviolet light, to cause the photocatalytic agent to undergo a photocatalytic reaction under the ultraviolet light, which causes destruction of micro-organisms on the ophthalmic lens.

### FIELD

The subject matter herein generally relates to a method for sterilizing an ophthalmic lens.

### BACKGROUND

Contact lenses are commonly worn by users to correct vision, or for cosmetic or therapeutic reasons. Each contact lens after hydration is usually placed in a polypropylene cup containing a care solution. The polypropylene cup is then sealed by an aluminum film. When a number of such polypropylene cups after packaging are collected, the polypropylene cups are placed in an autoclave for moist heat sterilization, which should cause destruction of micro-organisms on the contact lenses.

However, it usually takes a long time (one hour or two hours) for performing the moist heat sterilization. Furthermore, micro-organisms propagate rapidly when the contact lens is immersed in an aquatic environment, especially after a number of such polypropylene cups after package are collected. Also, the sealing interface between the polypropylene cup and the aluminum film may split at high temperature during the moist heat sterilization. Improvement in the art is preferred.

### BRIEF DESCRIPTION OF THE DRAWINGS

Implementations of the present disclosure will now be described, by way of example only, with reference to the attached figures.

FIG. 1 is a flowchart of an exemplary embodiment of a method for sterilizing an ophthalmic lens.

FIG. 2 is a diagram of a container used in the method of FIG. 1.

FIG. 3 is a diagram showing the ophthalmic lens placed in the container of FIG. 2.

FIG. 4 is a diagram showing the container of FIG. 3 exposed to ultraviolet light for sterilization.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to illustrate details and features of the present disclosure better.

The disclosure is illustrated by way of example and not by way of limitation in the figures of the accompanying drawings, in which like references indicate similar elements. It should be noted that references to “an” or “one” embodiment in this disclosure are not necessarily to the same embodiment, and such references mean “at least one.”

The term “comprising,” when utilized, means “including, but not necessarily limited to”; it specifically indicates open-ended inclusion or membership in the so-described combination, group, series, and the like.

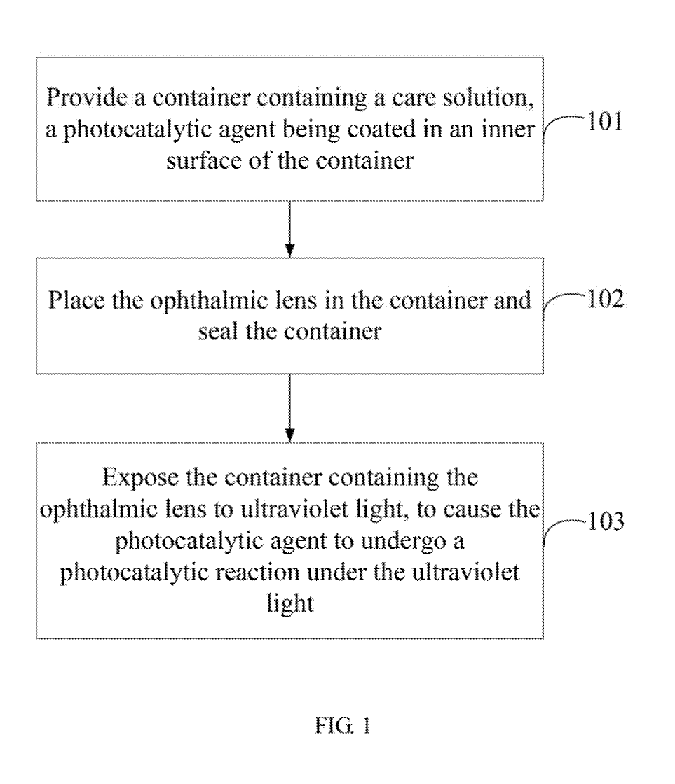

FIG. 1 illustrates a flowchart of an exemplary embodiment for a method for sterilizing an ophthalmic lens. The exemplary method is provided by way of example, as there are a variety of ways to carry out the method. Each block shown in the figure represents one or more processes, methods, or subroutines, carried out in the exemplary method. Furthermore, the illustrated order of blocks is by example only, and the order of the blocks can change. Additional blocks may be added or fewer blocks may be utilized, without departing from this disclosure. The exemplary method can begin at block 101.

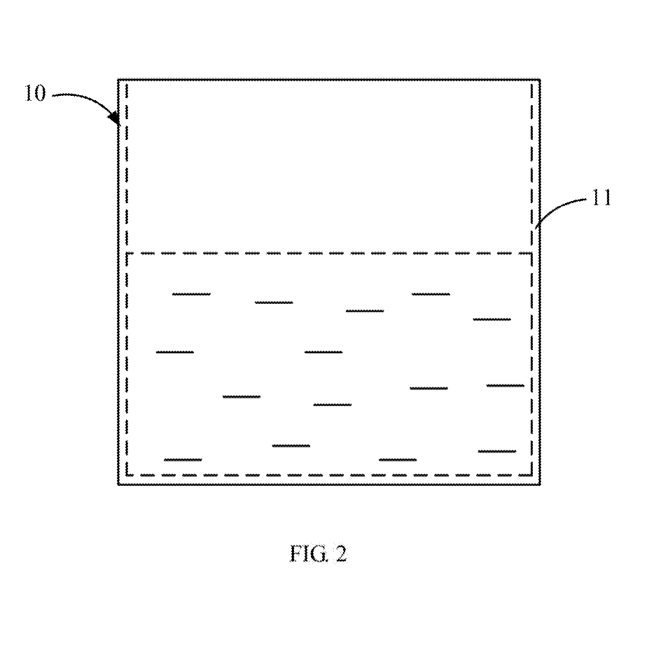

At block 101, referring to FIG. 2, a container 10 containing a care solution is provided. A photocatalytic agent 11 is coated in an inner surface of the container 10.

In at least one exemplary embodiment, the container 10 is a polypropylene cup. The photocatalytic agent 11 is nanosized particles of titanium dioxide (TiO₂).

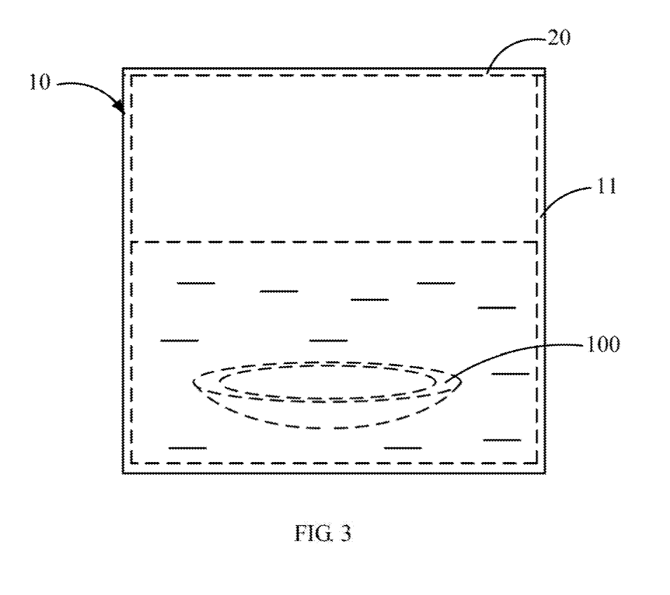

At block 102, referring to FIG. 3, the ophthalmic lens 100 is placed in the container 10, and the container 10 is sealed by an aluminum film 20.

In at least one exemplary embodiment, the ophthalmic lens 100 is a contact lens after hydration.

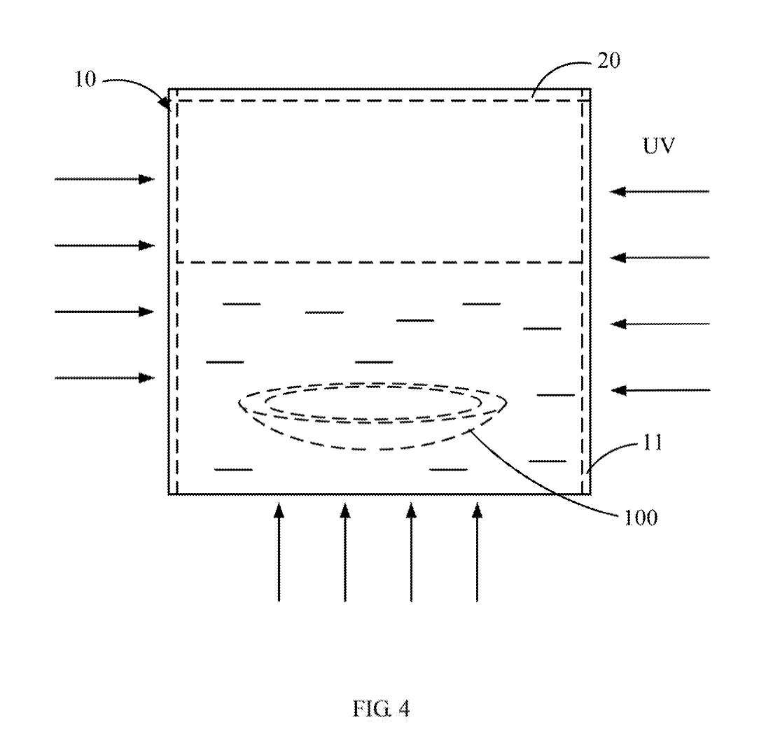

At block 103, referring to FIG. 4, the container 10 containing the ophthalmic lens 100 is exposed to ultraviolet light, to cause the photocatalytic agent 11 to undergo a photocatalytic reaction under the ultraviolet light, which causes destruction of micro-organisms on the ophthalmic lens 100.

Taking the TiO₂ for example, the photocatalytic reaction is carried out as follows:

Electron and hole pairs are generated when the ultraviolet light strikes the surface of the TiO₂. The electron and hole pairs respectively reduce and oxidize adsorbates on the surface, thereby producing radical species such as hydroxyl icons (.OH) and radical superoxide icons (.O₂). The hydroxyl icons and the radical superoxide icons can destroy micro-organisms on the ophthalmic lens 100. Because of its bandgap, TiO₂ requires ultraviolet light with less than 387.5 nm wavelength for the photocatalytic reaction.

In at least one exemplary embodiment, the ultraviolet light has a wavelength less than 387.5 nm.

The above method only takes several seconds to several minutes for sterilization. Thus, it takes less time compared to the moist heat sterilization. Furthermore, it can prevent the generation of micro-organisms for a long period of time. Also, since the method does not need high temperature, it can prevent the sealing interface between the container 10 and the aluminum film 20 from splitting. Finally, an ultraviolet illumination device is cheaper than the autoclave.

Depending on the embodiment, certain of the steps of methods hereinbefore described may be removed, others may be added, and the sequence of steps may be altered. It is also to be understood that the description and the claims drawn to a method may include some indication in reference to certain steps. However, the indication used is only to be viewed for identification purposes and not as a suggestion as to an order for the steps.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. A method for sterilizing an ophthalmic lens, comprising:
providing a container containing a care solution, wherein a photocatalytic agent is coated in an inner surface of the container;
placing the ophthalmic lens in the container and sealing the container; and
exposing the container containing the ophthalmic lens to ultraviolet light, to cause the photocatalytic agent to undergo a photocatalytic reaction under the ultraviolet light, which causes destruction of micro-organisms on the ophthalmic lens.

2. The method of claim 1, wherein the photocatalytic agent is nanosized particles of titanium dioxide.

3. The method of claim 2, wherein the ultraviolet light has a wavelength less than 387.5 nm.

4. The method of claim 1, wherein the container is a polypropylene cup.

5. The method of claim 1, wherein the container is sealed by an aluminum film.

6. The method of claim 1, wherein the ophthalmic lens is a contact lens after hydration.

7. A method for sterilizing an ophthalmic lens, comprising:
providing a container, wherein a photocatalytic agent is coated in an inner surface of the container;
placing the ophthalmic lens in the container and sealing the container; and
exposing the container containing the ophthalmic lens to ultraviolet light, to cause the photocatalytic agent to undergo a photocatalytic reaction under the ultraviolet light, which causes destruction of micro-organisms on the ophthalmic lens.

8. The method of claim 7, wherein the photocatalytic agent is nanosized particles of titanium dioxide.

9. The method of claim 8, wherein the ultraviolet light has a wavelength less than 387.5 nm.

10. The method of claim 7, wherein the container is a polypropylene cup.

11. The method of claim 7, wherein the container is sealed by an aluminum film.

12. The method of claim 7, wherein the ophthalmic lens is a contact lens after hydration.
